(19) United States
(12) Patent Application Publication
(10) Pub. No.: US 2016/0157779 A1
(43) Pub. Date: Jun. 9, 2016

(54) WEARABLE SENSOR APPARATUS WITH MULTIPLE FLEXIBLE SUBSTRATES

(71) Applicant: Intel Corporation, Santa Clara, CA (US)

(72) Inventors: Amit S. Baxi, Thane (IN); Vincent S. Mageshkumar, Navi Mumbai (MH); Kumar Ranganathan, Bangalore (IN)

(21) Appl. No.: 14/563,801

(22) Filed: Dec. 8, 2014

(51) Int. Cl. A61B 5/00; A61B 5/1477; A61B 5/11; A61B 5/0492; A61B 5/0408; A61B 5/0205

(52) U.S. Cl. CPC A61B 5/6831; A61B 5/0059; A61B 5/04085; A61B 5/02055; A61B 5/4266; A61B 5/11; A61B 5/0492; A61B 5/1477; A61B 5/6828; A61B 5/6823; A61B 5/6822; A61B 5/6824; A61B 5/6829; A61B 5/0002; A61B 5/0533

(57) ABSTRACT

Embodiments of the present disclosure provide techniques and configurations for a wearable sensor apparatus. In one instance, the apparatus may comprise a first flexible substrate, including a first plurality of sensors disposed on a first side of the first flexible substrate to be in direct contact with a user's body, and a first set of conductive connectors disposed on a second side of the first flexible substrate; and a second flexible substrate that includes a second set of conductive connectors disposed on a first side of the second flexible substrate compatibly to the first set of conductive connectors to mechanically and electrically couple the second flexible substrate with the first flexible substrate, and circuitry disposed on a second side of the second flexible substrate to receive and process readings provided by the sensors via the first and second sets of conductive connectors. Other embodiments may be described and/or claimed.

### FIELD

Embodiments of the present disclosure generally relate to the field of sensor devices, and more particularly, to wearable sensor devices with multiple flexible substrates that may be conformal with human body.

### BACKGROUND

With advances in various technologies, wearable sensing devices or systems are increasingly popular. However, today's wearable sensing devices or systems often fail to have a number of desirable attributes associated with them. A wearable sensing system may need to be comfortably attached to the human body, yet without slippage. However, the quality of signals sensed by a wearable system is highly dependent on the quality of contact with the human body, contrary to the need to provide comfort. Because a wearable sensing system may be in continuous contact with the human body, it may also need to be easily cleanable, preferably using conventional methods, such as a washing machine, for example. Furthermore, a wearable sensing system may need to have a desired sensing coverage, e.g., have an ability to effectively sense around movable spots of the human body, such as joints, wrists, fingers, ankles, and the like.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is a block diagram illustrating an example wearable sensor apparatus incorporated with the teachings of the present disclosure, in accordance with some embodiments.

FIG. 2 is a schematic diagram illustrating another view of the wearable sensor apparatus of FIG. 1, in accordance with some embodiments.

FIG. 3 is a schematic diagram of an architectural view of an example implementation of the digital front end node of FIGS. 1 and 2, in accordance with some embodiments.

FIG. 4 illustrates perspective views of various implementations of the digital front end node, in accordance with some embodiments.

FIG. 5 illustrates an example body area network formed by application of multiple wearable sensor apparatuses to a user's body, in accordance with some embodiments.

FIGS. 6-9 illustrate different views of an example wearable sensor apparatus that may be applied to a user's chest, in accordance with some embodiments.

FIGS. 10-11 illustrate different views of an example wearable sensor apparatus that may be applied to a user's knee, in accordance with some embodiments.

FIG. 12 is a process flow diagram for assembling a wearable sensor apparatus of the present disclosure, in accordance with some embodiments.

FIG. 13 illustrates an example computing device 1300 suitable for use with various components of FIG. 1, such as wearable sensor apparatus 100 including digital front end node or an external device of FIG. 1, in accordance with various embodiments.

### DETAILED DESCRIPTION

Embodiments of the present disclosure include techniques and configurations for a wearable sensor apparatus. In accordance with embodiments, the apparatus may comprise a first flexible substrate, including a plurality of sensors disposed on a first side of the first flexible substrate to be in direct contact with a user's body, and a first set of conductive connectors disposed on a second side of the first flexible substrate. The apparatus may further comprise a second flexible substrate that includes a second set of conductive connectors disposed on a first side of the second flexible substrate compatibly to the first set of conductive connectors to mechanically and electrically couple the second flexible substrate with the first flexible substrate. The apparatus may further comprise circuitry disposed on a second side of the second flexible substrate to receive and process readings provided by the sensors via the first and second sets of conductive connectors.

In the following detailed description, reference is made to the accompanying drawings that form a part hereof, wherein like numerals designate like parts throughout, and in which are shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical, electrical, or optical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or more elements are in direct contact.

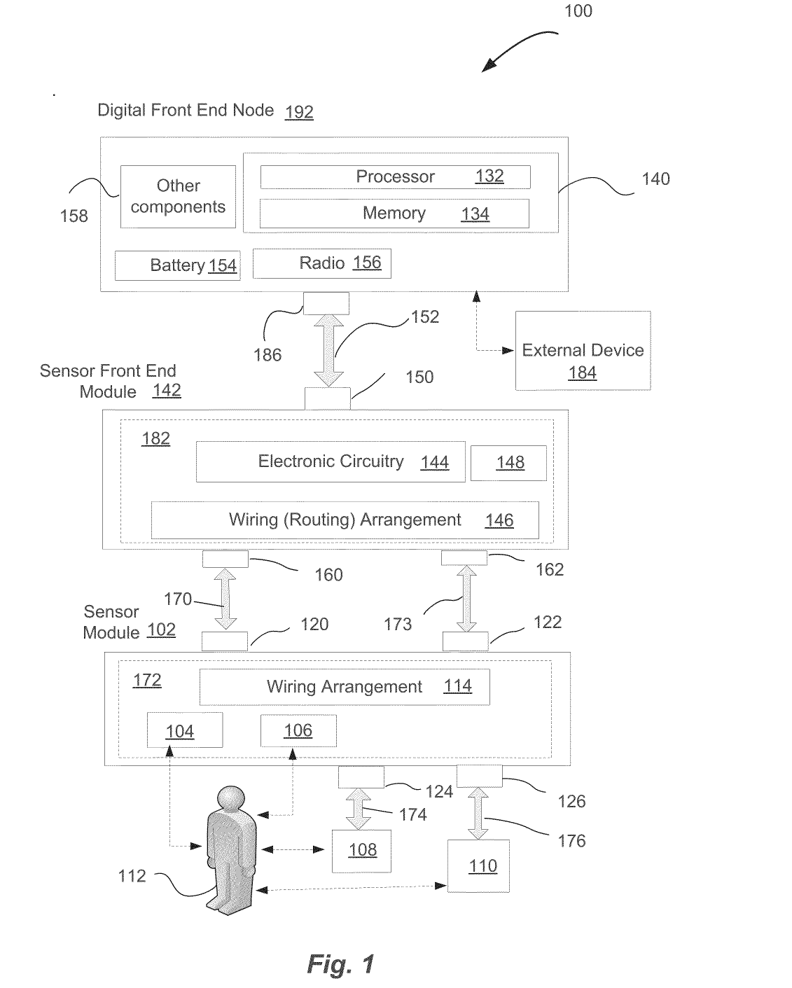

FIG. 1 is a block diagram illustrating an example wearable sensor apparatus 100 incorporated with the teachings of the present disclosure, in accordance with some embodiments. The apparatus 100 may comprise a sensor module 102 configured to be attachable to a user's body in order to conduct measurements associated with the functioning of the user's body and user's activities. In embodiments, the sensor module 102 may comprise a flexible substrate 172 (indicated by a dashed line) that may take different shapes and/or sizes, such as a strap, a band, or the like. The sensor module 102 may include a first plurality of sensors 104, 106 that may be disposed to be in direct contact with a user's body 112. For example, the sensors 104, 106 may be placed around an inner side of the flexible substrate 172 of the sensor module 102, to provide direct contact with the user's body 112. In some embodiments, the sensors 104, 106 may be built in (e.g., embedded) in the inner side of the flexible substrate 172 of the sensor module 102. The sensors 104, 106 may provide readings related to various user body functions as discussed below in greater detail. The sensors 104, 106 may be resistant to damage due to stretching of the flexible substrate, washing of the flexible substrate, and the like.

The sensor module 102 may further include a set of conductive connectors 120, 122 that may be disposed on an outer side of the flexible substrate 172 of the sensor module 102. The conductive connectors 120, 122 may comprise flexible damage-resistant patches having conductive properties, and may provide dual functionality of electric and mechanical coupling (indicated by arrows 170, 173) with a sensor front end module 142, as will be described below in greater detail. The conductive connectors 120, 122 may be made of elastic fabric, elastomer, polymer, or other suitable materials.

The sensor module 102 may further include a second plurality of sensors 108, 110, also disposed on the inner side of the flexible substrate 172 of the sensor module 102 to provide direct contact with the user's body 112. The second plurality of sensors 108, 110 may include sensors that may not be damage- or water-resistant (e.g., washable). Accordingly, the sensors 108, 110 may be detachably coupled, mechanically and electrically, with the sensor module 102 via another set of conductive connectors 124, 126, as indicated by arrows 174, 176.

It should be noted that sensors 104, 106 of the first plurality of sensors and sensors 108, 110 of the second plurality of sensors are shown in FIG. 1 for illustration only and are not limiting the implementation of apparatus 100. It will be appreciated that any number of sensors or conductive connectors similar to 120, 122 may be used in the apparatus 100.

The sensor module 102 may further include a wiring arrangement 114 to provide electric connections for the sensors 104, 106, 108, 110 with conductive connectors 120 and 122. The wiring arrangement 114 may be built in (e.g., embedded, embroidered, woven, imprinted, and the like) the flexible substrate 172 of the sensor module 102, as will be described below in greater detail. The wiring arrangement 114 may be damage resistant, e.g., in response to flexing, stretching, or washing of the flexible substrate 172.

The apparatus 100 may further include a sensor front end module 142 that may be electrically and mechanically connectable with the sensor module 102. The sensor front end module 142 may comprise a flexible substrate 182 (indicated by a dashed line) having a shape that may be compatible with or conformable to the shape of the flexible substrate 172 of the sensor module 102. The sensor front end module 142 may include a set of conductive connectors 160, 162 disposed on an inner side of the flexible substrate 182 compatibly to the conductive connectors 120, 122. In other words, conductive connectors 160, 162 may be disposed to mate the conductive connectors 120, 122 to mechanically and electrically couple the sensor front end module 142 with the sensor module 102.

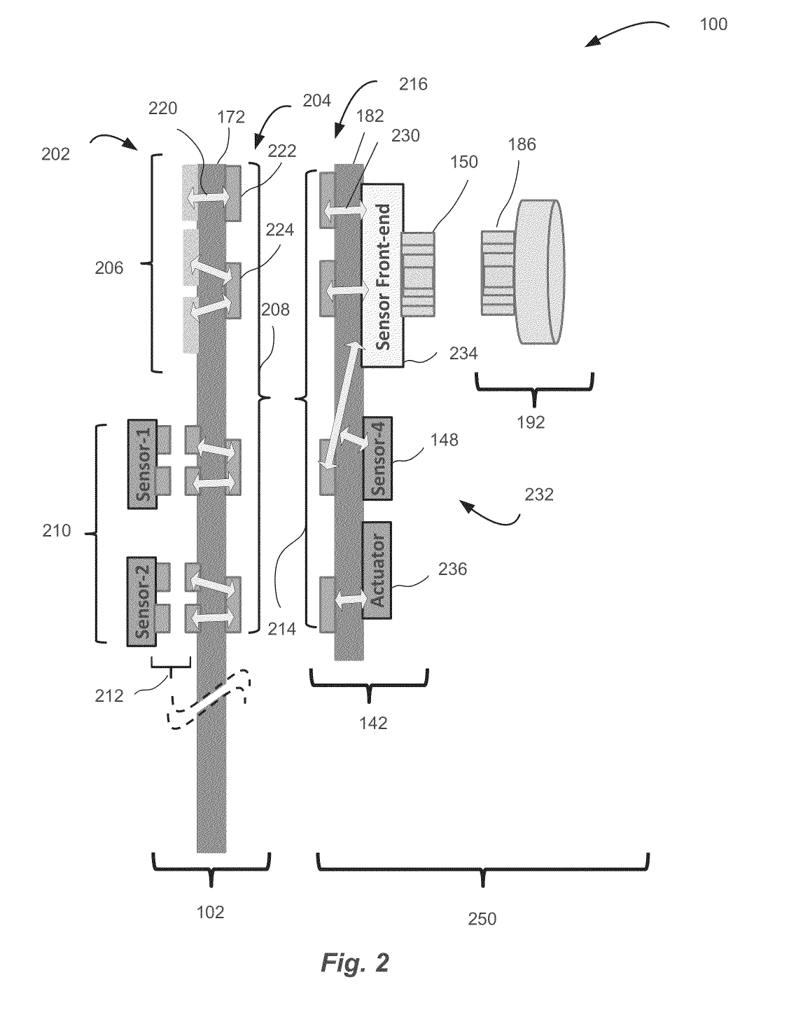

The sensor front end module 142 may further include electronic circuitry 144 configured to receive and process readings provided by the sensors 104, 106 via the wiring arrangement 114, the conductive connectors 120, 122, and corresponding conductive connectors 160, 162. The circuitry 144 may be further configured to provide power and excitation to the sensors, transduce the sensor signals into voltage, amplify and condition the sensor signals. The circuitry 144 may be further configured to receive and pre-process readings provided by the sensors 108, 110 via the conductive connectors 124, 126, wiring arrangement 114, conductive connectors 120, 122, and corresponding conductive connectors 160, 162. In embodiments, the electronic circuitry 144 may be disposed on an outer side of the flexible substrate 182. The functionality of the electronic circuitry 144 will be described in greater detail in reference to FIG. 2.

The sensor front end module 142 may further include a wiring arrangement 146 to provide routing of electric signals (sensor readings from sensors 104, 106, 108, 110) coming through the conductive connectors 160, 162, to the electronic circuitry 144. The wiring arrangement 146 may be configured similar to wiring arrangement 114, e.g., it may be built in the flexible substrate 182. In some embodiments, the sensor front end module 142 may include one or more sensors 148 that may be coupled with the flexible substrate 182.

The sensor front end module 142 may further include an electric connector (e.g., multi-pin contact) 150 to provide mechanical and electric coupling 152 with a digital front end node 192. The digital front end node 192 may be configured to further process the readings provided by the sensors 104, 106, 108, 110 of the sensor module 102 and sensors 148 of the sensor front end module 142.

In some embodiments, the digital front end node 192 may include a processing unit 140 having a processor 132 configured to process the readings (signals) provided by the sensors 104, 106, 108, 110, 148. The processing unit 140 may include memory 134 having instructions that, when executed on the processor 132, may cause the processor 132 to perform signal processing. The digital front end node 192 may include a battery 154 configured to provide power supply to the digital front end node 192 and, more generally, to the apparatus 100. The digital front end node 192 may include a radio 156 to transmit processed data resulting from processing the sensor readings for further processing, e.g., to an external device 184 (e.g., mobile or stationary computing device). The digital front end node 192 may include a mating connector 186 to mate the connector 150 of the sensor front end module 142.

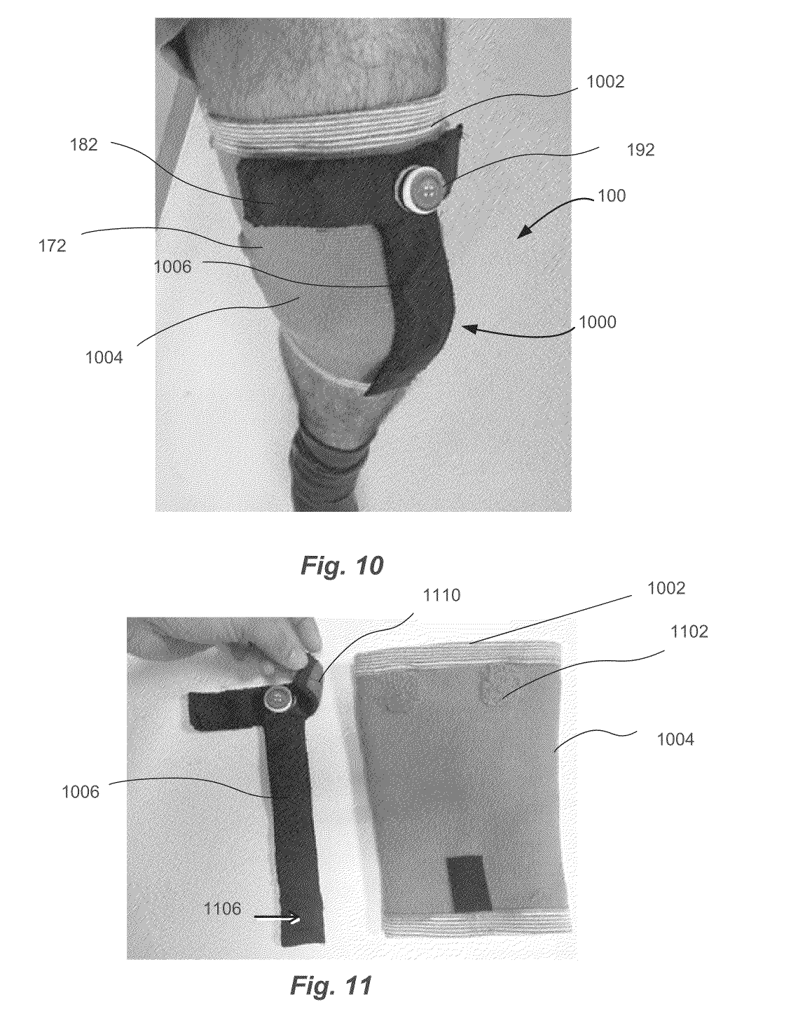

The digital front end node 192 may include other components 158 necessary for the functioning of the apparatus 100, as described in greater detail in reference to FIGS. 10 and 11. For example, the processor 132, memory 134, and other components of the digital front end node 192 may be coupled with one or more interfaces (not shown) via radio 156 in order to facilitate information exchange among the above-mentioned components. Communications interface(s) (not shown) may provide an interface for the apparatus 100 to communicate over one or more wired or wireless network(s) and/or with any other suitable device, such as external device 184.

FIG. 2 is a schematic diagram illustrating another view of the example wearable sensor apparatus 100 in accordance with some embodiments. As described in reference to FIG. 1, apparatus 100 may include a sensor module 102, mechanically and electrically coupled with sensor front end module 142. The sensor module 102 may comprise a flexible substrate (e.g., washable strap) 172 having a first (inner) side 202 and a second (outer) side 204. The flexible substrate 172 may be attached to a user's body (not shown) at the first side 202. Depending on the application, the flexible substrate 172 may be wrapped around different user body parts such as chest, knee, wrist, neck, etc., and may be adjusted to the right level of snugness using a simple attachment (e.g., Velcro®) arrangement.

As described above, a first plurality of damage-resistant sensors 206 (e.g., 104, 106 of FIG. 1) may be disposed around the first side 202 to be in direct contact with the user's body. In some embodiments, the first plurality of sensors 202 may be built in the first side 202 of the flexible substrate 172 of the sensor module 102. In embodiments, the first plurality of sensors 202 may include optical photodiodes, electrocardiogram (ECG) electrodes, electromyogram (EMG) electrodes, galvanic skin response (GSR) electrodes, piezo crystals, pressure sensors, stretch sensors, or the like.

The flexible substrate 172 may further include a set of conductive connectors 208 (similar to 120, 122 of FIG. 1) that may be disposed on the second side 204 of the flexible substrate 172 of the sensor module 102. The set of conductive connectors 208 may comprise flexible damage-resistant patches, such as Velcro® conductive patches having conductive properties, and may provide dual functionality of electric and mechanical coupling with the sensor front end module 142. As shown, the set of conductive connectors 208 may include multiple conductive Velcro® patches as flexible connectors between the sensor module 102 and sensor front end module 142 to maintain conformability, while also providing washability, as well as scalability to add more sensors as needed, depending on the wearable apparatus 100's application.

The sensor module 102 may further include a second plurality of sensors 210 (similar to 108, 110 of FIG. 1) disposed on the first side 202 of the flexible substrate 172. The second plurality of sensors 210 may include sensors that may not be damage- or water-resistant (e.g., washable), such as temperature sensors, sweat chemical sensors, optical sensors, or the like. More generally, the second plurality of sensors may include a variety of washable sensors configured to provide readings associated with user's health state. Accordingly, the second plurality of sensors 210 may be mechanically and electrically coupled with the flexible substrate 172 by using a corresponding set of conductive connectors (e.g., Velcro® conductive patches) 212.

The sensor module 102 may further include a wiring arrangement 220 (schematically indicated by arrows) to provide electric connections for the sensors 206 and 210 with conductive connectors 208. The wiring arrangement 220 may be built in (e.g., embedded, embroidered, woven, imprinted, and the like) the flexible substrate 172. For example, electrical connections may be made by screen printing the traces on the flexible substrate 172 from sensors 206 and/or 210 to conductive connectors 212 using conductive inks.

In another example, a meandering (e.g., sine wave type, zig-zag shaped) pattern of traces (e.g., copper traces) deposited on a plastic substrate (e.g., polyamide) may be used. This plastic substrate may be moulded with the flexible substrate 172 or attached to the flexible substrate 172 using a waterproof adhesive. Conductive glue may be applied to make electrical connection from sensors 206 and connectors 212 to the copper traces. In another example, insulated ultra-thin (e.g., copper or silver) wires moulded with the flexible substrate 172 in a meandering pattern may be used to provide desired electrical connections.

As discussed above, it may be desirable to have the wiring arrangement 202 on flexible substrate 172 be damage-resistant, such as washable. Hence, it may be preferable to minimize the extent of the wiring arrangement 220 on the flexible substrate 172. For example, the wiring arrangement 220 may be minimized or even eliminated by placing the sensors 206 and/or 210 around the substrate 172 where the sensor signals may be picked up, to optimize signal pickup. For example, ECG electrodes of the sensors 206 may be placed on the flexible substrate 172 on either side of the center of the chest of the user's body and separated by at about 10 cm to pick up the desired ECG signal. GSR electrodes may provide readings of desired strength when placed on portions of the flexible substrate 172 that correspond to sides of the user's body. Temperature sensors of sensors 210 may provide signals of desired strength when placed on the flexible substrate 172 around the user's armpit.

The flexible conductive connectors 208 may be placed as close to the sensors 206 and/or 210 as possible, for example, substantially directly across from (on the other side of) the sensors, as illustrated by the placement of the plurality of sensors 206 and corresponding placement of the conductive connectors 222 and 224 of the set of conductive connectors 208. In summary, it may be desirable to distribute flexible conductive connectors 208 around the washable flexible substrate 172, rather than make extensive use of the wiring arrangement 220.

As described above, the sensor front end module 142 may comprise the flexible substrate 182 having a shape that may be compatible with or conformable to the shape of the flexible substrate 172 of the sensor module 102, to enable conformability and disaggregation of the apparatus 100. The flexible substrate 182 may include a set of conductive connectors 214 (similar to 160, 162 of FIG. 1) disposed on a first (inner) side 216 of the flexible substrate 182 to mate the conductive connectors 208 to mechanically and electrically couple the sensor front end module 142 with the sensor module 102. Thus, conductive connectors 208 and corresponding conductive connectors 214 enable mechanical attachment of the sensor module 102 to sensor front end module 142 and provide electric connections between sensor front end module 142 and sensors 206 and 210 distributed on sensor module 102.

Necessary wiring and signal routing may be provided by the wiring arrangement (schematically indicated by arrows 230) inside the flexible substrate 182, once the sensor signal is transferred from the washable flexible substrate 172 to non-washable flexible substrate 182 via the wiring arrangement 220 and conductive connectors 208 and 214. As shown, the sensor signal routing may be provided to connect the conductive connectors 214 with components of electronic circuitry disposed on a second (outer) side 232 of the flexible substrate 182 and briefly described in reference to electronic circuitry 144 of FIG. 1.

In embodiments, electronic circuitry components may include sensor front end 234 and actuator 236 coupled with the plurality of sensors 206. As discussed above, some examples of sensors 206 may include optical photodiodes providing current output, ECG electrodes providing bio-potential output, piezo crystals providing charge output, pressure sensors providing capacitance output, stretch sensors providing resistance change output, and the like. In summary, the sensors 206 may provide output in different forms (e.g., voltage, current, resistance change, etc.). These forms of output may be converted to a voltage before further processing, e.g., feeding the converted voltages to an analog to digital converter (ADC) for digitization. The sensor front end 234 may be configured to perform that conversion. Moreover, sensors 206 may provide a signal having strength below the desired threshold, which may need to be amplified, conditioned, and filtered before it may be digitized by the ADC. The sensor front end 234 may also include the required mixed-signal circuits to transduce, amplify, and condition the signal before feeding it to the digital front end node 192 for digitization. The sensor front end 234 may power (electrically excite) the sensors 206, 148, and actuator 236. The actuator 236 may be used to provide haptic feedback to the user (e.g. using a micro-vibrator).

As described in reference to FIG. 1, the sensor front end module 142 may include one or more sensors 148. The sensors 148 may include different types of sensors, such as temperature sensors, respiration sensors, and the like. It will be appreciated that sensors 148 may not be damage-resistant and accordingly may be attached to the non-washable flexible substrate 182.

As also described in reference to FIG. 1, the sensor front end 234 may be connectable (via multi-pin connector 150 and mating connector 186) with the digital front end node 192. The digital front end node 192 may be configured to process the readings (sensor signals) pre-processed (e.g., amplified, conditioned, and filtered) by the sensor front end 234.

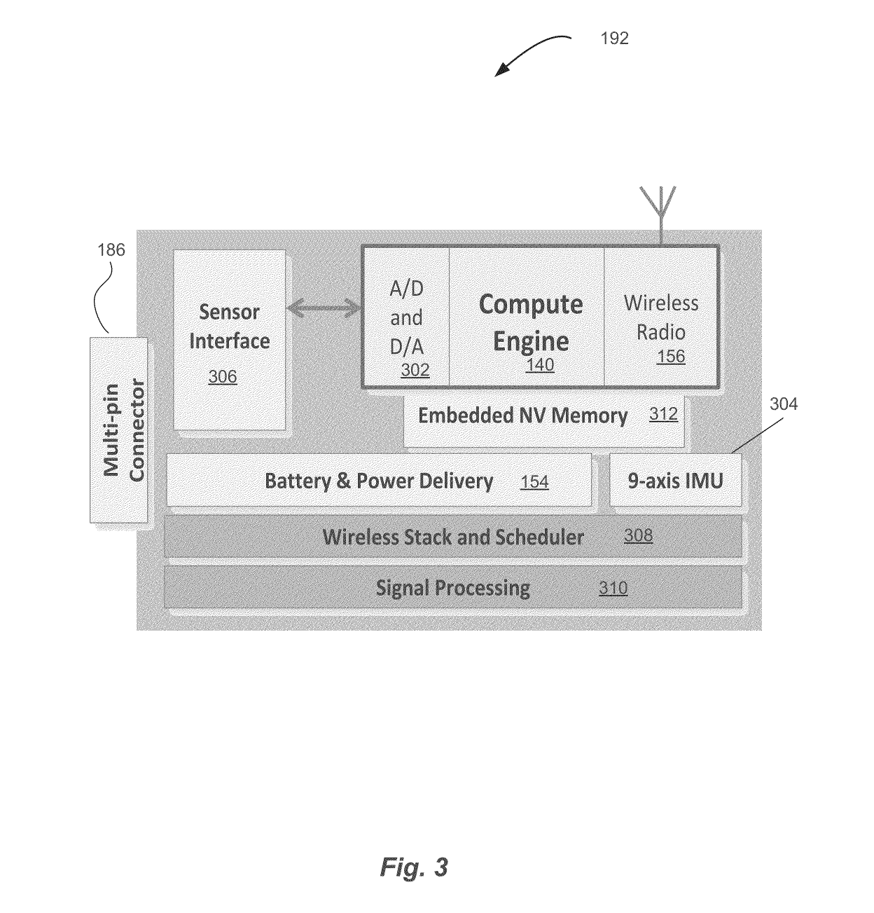

FIG. 3 is a schematic diagram of an architectural view of an example implementation of the digital front end node 192, in accordance with some embodiments. As described in reference to FIG. 1, the digital front end node 192 may integrate the battery 154, local storage memory 312 for storing and forwarding sensor data, compute engine (e.g., processing unit 140), wireless radio 156, and other components (referenced in FIG. 1 under numeral 158), with multiple types of sensors across the wearable sensor apparatus 100.

The other components of the digital front end node 192 may include an analog to digital and digital to analog (A/D and D/A) convertors 302 to digitize and de-digitize sensor signals provided by the sensor front end 234. The other components may further include an inertial measurement unit (IMU) 304 (e.g., a 9-degree of freedom IMU comprising a magnetometer, gyroscope, and accelerometer) to cancel noise and motion artifact effects for any type of wearable sensor apparatus 100 (e.g., located at chest, knee, wrist, etc.). The other components of the digital front end node 192 may further include a sensor interface 306, wireless stack and scheduler 308, and signal processing module 310. The components 306, 308, and 310 may comprise software solutions and may be stored in the memory of the processing unit 140 or memory 312. Digital front end node 192 may be pluggable into the sensor front end 234 by means of a miniature multi-pin connector 186.

In summary, the digital front end node 192 may be configured to supply power to both sensor module 102 and sensor front end module 142, and perform data acquisition and closed loop system control. The digital front end node 192 may be further configured to perform signal de-noising, feature extraction, classification, data compression, and wireless transmission of sensed signals over a network (e.g., local wireless network).

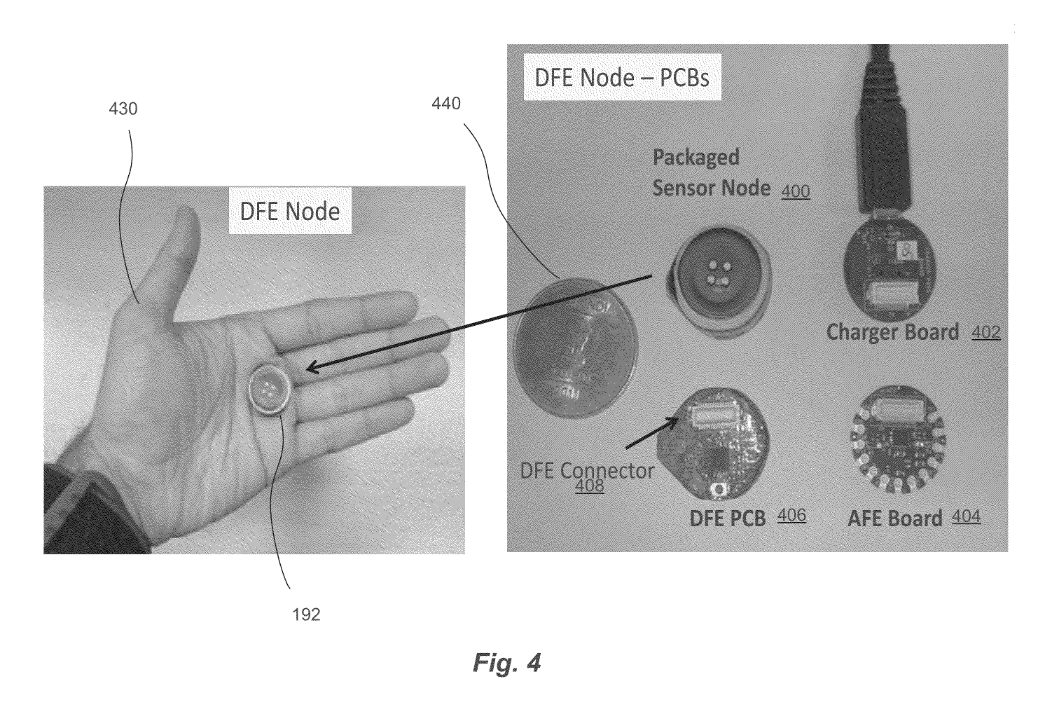

FIG. 4 illustrates perspective views of various example implementations of the digital front end node 192, in accordance with some embodiments. As illustrated, the digital front end node 192 may be implemented as a miniature button-sized node 400 (as shown in a palm of a hand 430 and comparable in size to a coin 440. The digital node 400 may comprise a rechargeable coin cell battery (not shown here) packaged along with a Digital Front-End (DFE) printed circuit board (PCB) 406 having connector 408. The battery inside DFE Node 400 could be charged by plugging the DFE Node 400 on a separate charger board 402 and a micro-USB cable. The Sensor front-end 234 (in FIG. 2) could comprise of analog front end board (AFE) 404. The functionality of these components is described in reference to FIGS. 1-3.

Referring again to FIG. 2, the sensor front end module 142 and the digital front end node 192 may comprise a sensor node 250 that may be conformable with the sensor module 102. More specifically, in contrast to wearable conventional sensor solutions, the conformable sensor node 250 may comprise a conformable flexible substrate (e.g., strap) 182 and a miniature and generically designed digital front end node 192 that may be pluggable into the flexible substrate 182. This approach may result in a conformable, stretchable, and modular sensor node 250. The flexible substrate 182 of the sensor front end module 142 may have a desirable thickness, and may be flexible and conformal to assume essentially the same contour (e.g., to conform to a particular part of the user's body) as the sensor module 102, when the sensor module 102 and attached sensor node 250 wrap around the user's body or the body part.

A wearable sensor apparatus implemented as described above may provide a number of advantages compared to conventional wearable sensor solutions. For example, the apparatus described above may provide for a modular wearable system, in which a digital front end node may comprise a generic and sensor agnostic wireless node, connectable and attachable to different types of sensor front end modules, such as, for example, different flexible substrate straps. These flexible substrate straps may include a chest strap for heart rate sensing, knee strap for knee kinematics sensing, wrist strap for optical heart rate sensing, and the like. The digital front end node may be used without modification for multiple sensing applications. Separation of the sensor front end module and digital front end node may provide for a desired form factor of the digital front end node. The flexible substrate of the sensor front end module may be attachable to the flexible substrate of the sensor module at multiple points along the length of the sensor module flexible substrate, which may enable integration of multiple distributed sensors in the sensor module. Further, multiple sensors may be added and/or replaced to a flexible substrate of the sensor module because sensors may be plugged on or detached from the flexible substrate straps without compromising cleanability of the sensor module. Furthermore, conformal substrate straps may be integrated with (e.g., stitched on) existing garments to create smart garments, thereby reducing or eliminating the need to instrument large surfaces for placing sensors and simplifying the manufacturing process.

Use of conductive connectors (e.g., Velcro®) as flexible conductive connectors may provide for retaining flexibility without compromising modularity. It may also enable separation of the sensor front end module and pluggable sensors from the sensor module and enable cleanability (washability) of the sensor module. The digital front end node may be replaceable, e.g., along with a firmware upgrade to enable new system functionality. Furthermore, based on specific requirements associated with a particular use of the wearable sensor apparatus, different versions of a digital front end node may be designed to provide different levels of computing capabilities (i.e., different central processing units (CPUs)) and/or different radios for different wireless standards.

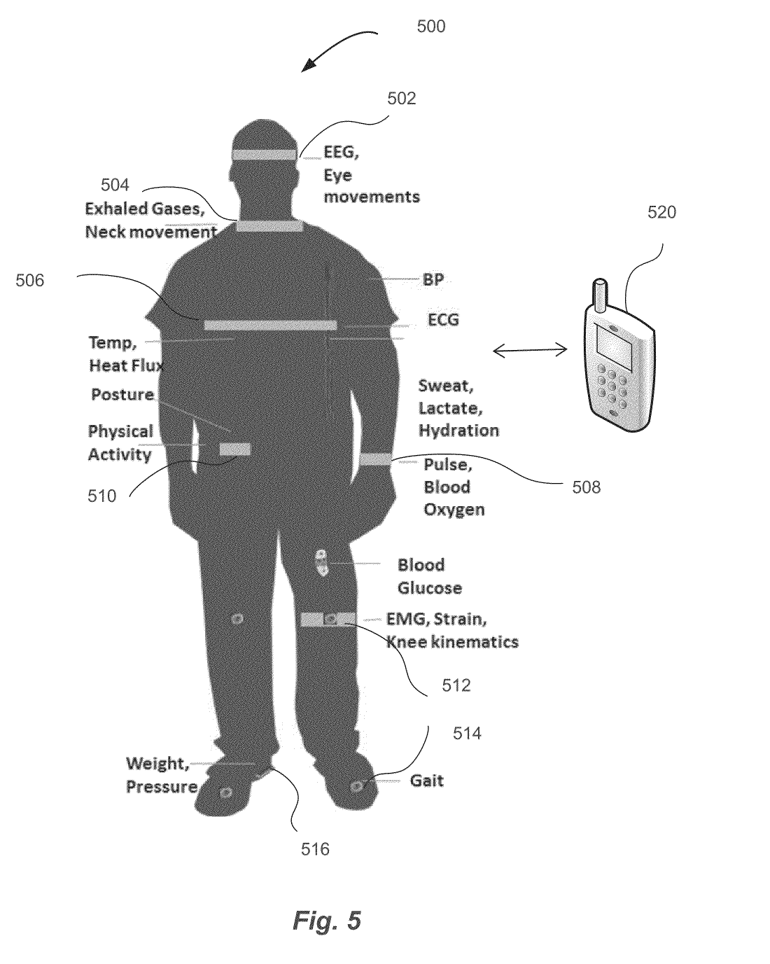

As described above, modular, conformal, washable wearable sensor apparatuses may be designed for different applications such as for monitoring ECG, respiration, gait analysis, heart rate, and the like. In some instances, multiple wearable sensor apparatuses may apply to a user's body to form a body area network, enabling a host of different applications. FIGS. 5-10 illustrate various examples of applications of a wearable sensor apparatus, in accordance with some embodiments.

FIG. 5 illustrates an example body area network formed by application of multiple wearable sensor apparatuses to a user's body, in accordance with some embodiments. As shown, the wearable sensor apparatus (e.g., 100) may be applied to different parts of a user's body 500, forming a body area network. For example, a wearable sensor apparatus 502 may be attached to a user's forehead to sense and measure eye movements, apnea, sleep, human-computer interaction (HCI) parameters, electroencephalography (EEG), electrooculography (EOG), and the like. A wearable sensor apparatus 504 may be attached to a user's neck to sense and measure a range of neck movement, breath parameters, or posture. A wearable sensor apparatus 506 may be attached to a user's chest to sense and measure temperature, posture, heat parameters, and kick count, fetal heart rate, and uterine contractions (for pregnant women).

A wearable sensor apparatus 508 may be attached to different parts of a user's arm, e.g., shoulder, wrist, or finger, to sense and measure sweat, lactate, hydration, blood pressure, oxygen, ECG, body fat, stress parameters, pulse, and the like. A wearable sensor apparatus 510 may be attached to a user's waist or abdomen to sense and measure posture or physical activity. A wearable sensor apparatus 512 may be attached to different parts of a user's leg, such as knee or ankle, to sense and measure electromyography (EMG), movement, inflammation, knee or joint kinematics, blood glucose, and the like. A wearable sensor apparatus 514 or 516 may be attached to various parts of a user's foot to sense and measure weight, pressure, gait, rehabilitation parameters, and the like. Data measured by the apparatuses 502-516 may be provided (e.g., via a wireless network) to an external device 520 (e.g., smartphone, tablet computer, or the like) for aggregation, processing, and display to the user.

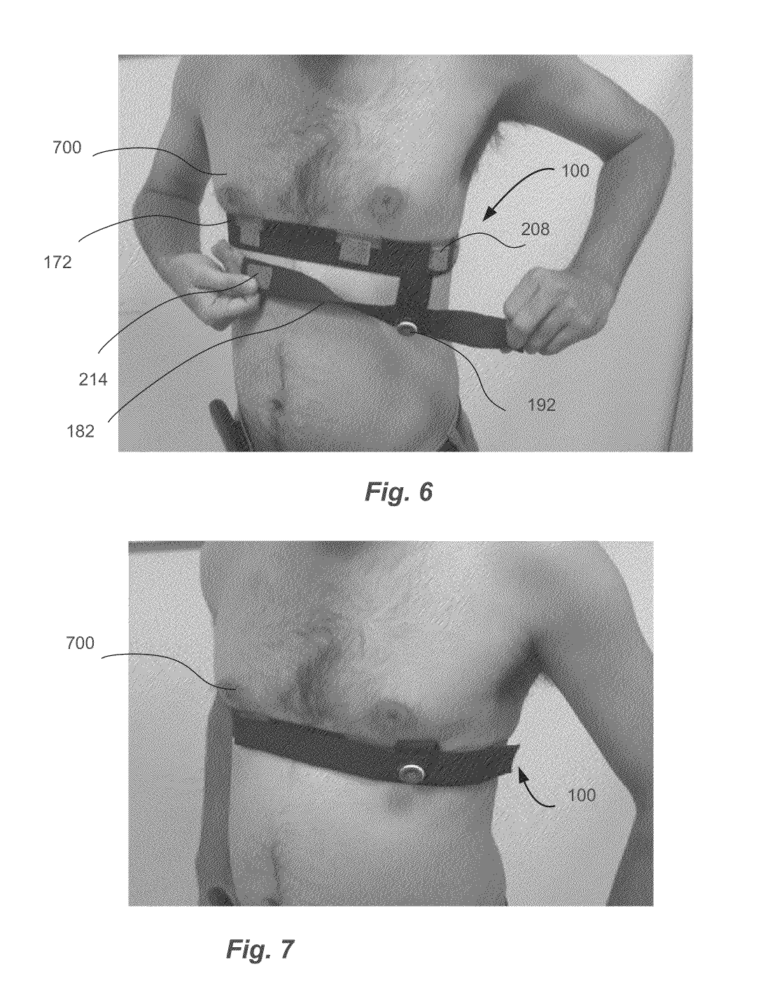

FIGS. 6-9 illustrate different views of an example wearable sensor apparatus that may be applied to a user's chest, in accordance with some embodiments. FIG. 6 illustrates an example application of an example wearable sensor apparatus 100 to the user's chest. As described above, the apparatus 100 may include the sensor module 102 comprising a flexible substrate (washable strap) 172 that is wrapped around the user's chest 700 as shown, and sensor front end module 142 comprising a flexible substrate (conformal strap) 182. The conductive connectors (flexible Velcro® patches) 208 and mating conductive connectors 214 are shown on the flexible substrate (washable strap) 172 and flexible substrate (conformal strap) 182. The digital front end node 192 is shown as plugged into the sensor front end module 142.

FIG. 7 illustrates the example wearable sensor apparatus 100 as assembled. As shown, flexible substrate (conformal strap) 182 is attached to flexible substrate (washable strap) 172 via conductive connectors 208 and 214) and the assembled wearable sensor apparatus 100 is attached to the user's chest 700. The wearable sensor apparatus 100 may include a number of sensors (not shown), such as ECG and GSR (comprising conductive fabric electrodes) embedded in the flexible substrate (washable strap) 172, respiration sensors (e.g., stretch sensors) and temperature sensors attached to the flexible substrate (conformal strap) 182, and IMU embedded in the digital front end node 192.

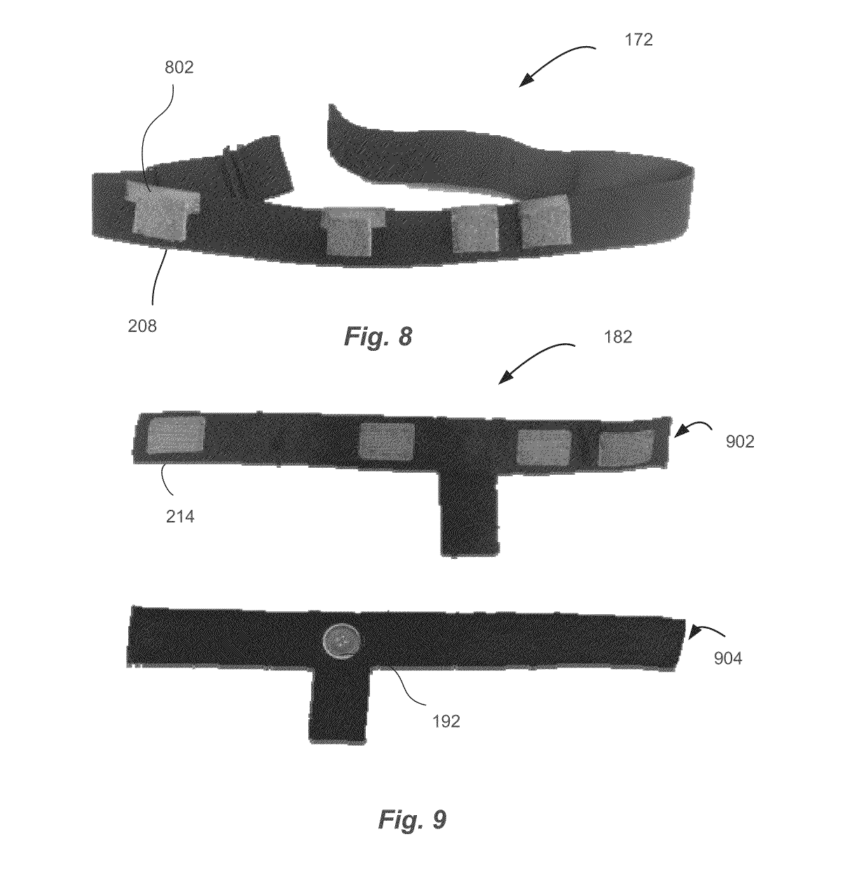

FIG. 8 illustrates an example flexible substrate (washable strap) 172 of the wearable sensor apparatus 100, shown at its outer side (corresponding to side 204 as referenced in FIG. 2). The conductive connectors (flexible Velcro® patches) 208 are shown on the flexible substrate (washable strap) 172. Portions 802 of the washable sensors 206 are also visible, although the washable sensors 206 are substantially disposed on the inner side (corresponding to side 202 of FIG. 2), which is not shown in FIG. 8, directly opposite the conductive connectors 208, in order to provide desired signal strength. The sensors 206 may include washable conductive fabric electrodes for ECG and GSR, for example.

FIG. 9 illustrates an example flexible substrate (conformal strap) 182 of the wearable sensor apparatus 100, shown at its inner side 902 (corresponding to side 216 as referenced in FIG. 2), and its outer side 904 (corresponding to side 232 as referenced in FIG. 2). As described above, the flexible substrate (conformal strap) 182 may include a plurality of conductive connectors 214, one or more sensors 148 (not shown in FIG. 9), and circuitry 144 (also not shown in FIG. 9) configured to pre-process readings provided by all sensors included in the apparatus 100.

FIGS. 10-11 illustrate different views of an example wearable sensor apparatus 100 (knee strap apparatus) that may be applied to a user's knee, in accordance with some embodiments. FIG. 10 illustrates the apparatus 100 wrapped around the user's knee 1000. The flexible substrate (washable strap) 172 may comprise a sensor-bearing strap 1002 and stretchable knee cap 1004. The flexible substrate (conformal strap) 182 may comprise a conformal knee strap 1006 attachable to knee cap 1004 via conductive connectors (flexible Velcro® patches, not shown). The digital front end node 192 may provide wireless connectivity, such as Bluetooth® low energy network for continuous data streaming to an external device (not shown).

FIG. 11 illustrates the apparatus 100 detached from the user's knee cap. As shown, the knee cap 1004 includes conductive connectors (flexible Velcro® patches) 1102. The inner side of the knee cap 1004 may include washable sensors, such as EMG electrodes (not shown), that may be disposed directly opposite the conductive connectors 1102, in order to provide desired signal strength. The conformal knee strap 1006 may include fabric-based stretch sensors embedded into the conformal knee strap 1006 (not shown) and IMU 1106 integrated into the conformal knee strap 1006. The fabric knee strap 1006 may include mating conductive connectors (flexible Velcro® patches) 1110 to match the conductive connectors 1102.

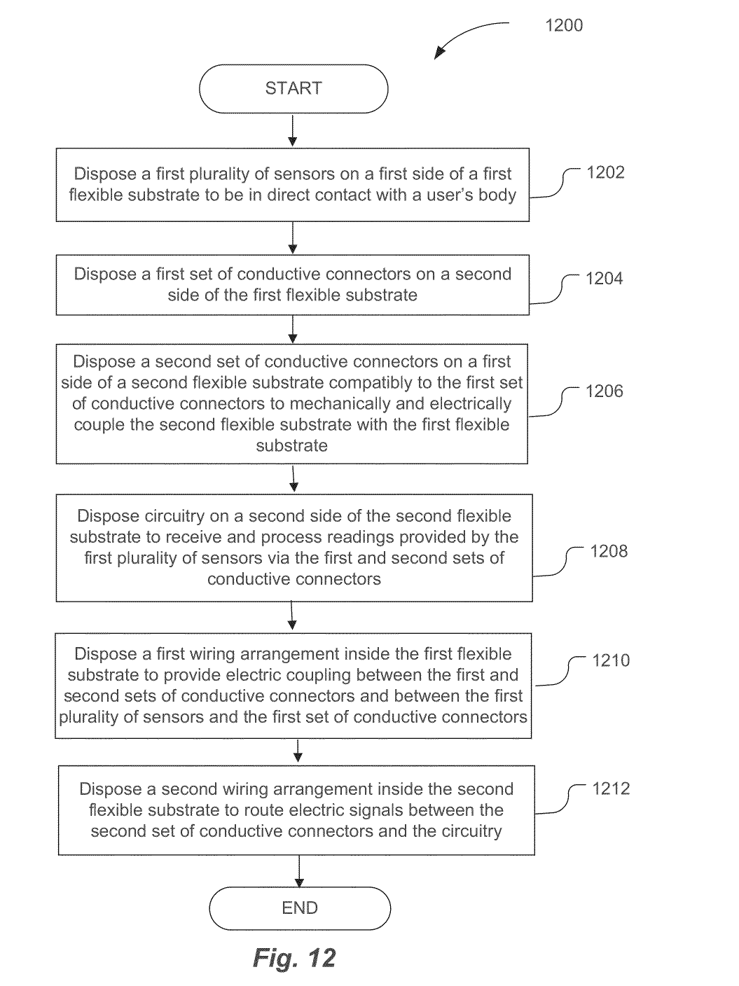

FIG. 12 is a process flow diagram for assembling a wearable sensor apparatus, in accordance with some embodiments. The process 1200 may comport with some of the apparatus embodiments described in reference to FIGS. 1-11. In alternate embodiments, the process 1200 may be practiced with more or less operations, or different order of the operations.

The process 1200 may begin at block 1202 and include disposing a first plurality of sensors on a first side of a first flexible substrate to be in direct contact with a user's body.

At block 1204, the process 1200 may include disposing a first set of conductive connectors on a second side of the first flexible substrate.

At block 1206, the process 1200 may include disposing a second set of conductive connectors on a first side of a second flexible substrate compatibly to the first set of conductive connectors to mechanically and electrically couple the second flexible substrate with the first flexible substrate. Additionally, the process 1200 may include disposing a third set of conductive connectors on the first side of the first flexible substrate and attaching a second plurality of sensors to the first side via the third set of conductive connectors.

At block 1208, the process 1200 may include disposing circuitry on a second side of the second flexible substrate to receive and process readings provided by the first plurality of sensors via the first and second sets of conductive connectors. The circuitry may be further configured to receive and process readings provided by the second plurality of sensors via the first, second, and third sets of conductive connectors.

At block 1210, the process 1200 may include disposing a first wiring arrangement inside the first flexible substrate to provide electric coupling between the first and second sets of conductive connectors and between the first plurality of sensors and the first set of conductive connectors.

At block 1212, the process 1200 may include disposing a second wiring arrangement inside the second flexible substrate to route electric signals between the second set of conductive connectors and the circuitry.

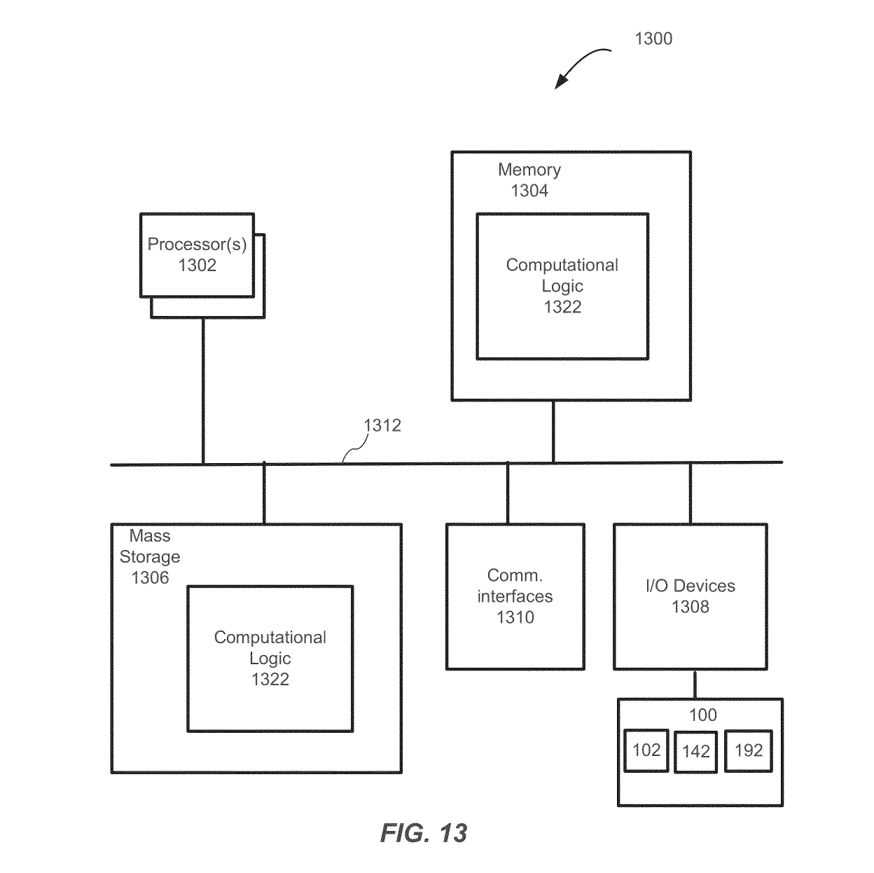

FIG. 13 illustrates an example computing device 1300 suitable for use with various components of FIG. 1, such as wearable sensor apparatus 100 including digital front end node 192 or an external device 184 of FIG. 1, in accordance with various embodiments. In some embodiments, various components of the example computing device 1300 may be used to configure the digital front end node 192. In some embodiments, various components of the example computing device 1300 may be used to configure the external device 184. As shown, computing device 1300 may include one or more processors or processor cores 1302 and system memory 1304. For the purpose of this application, including the claims, the terms “processor” and “processor cores” may be considered synonymous, unless the context clearly requires otherwise. The processor 1302 may include any type of processors, such as a central processing unit (CPU), a microprocessor, and the like. The processor 1302 may be implemented as an integrated circuit having multi-cores, e.g., a multi-core microprocessor. The computing device 1300 may include mass storage devices 1306 (such as solid state drives, volatile memory (e.g., dynamic random-access memory (DRAM), and so forth). In general, system memory 1304 and/or mass storage devices 1306 may be temporal and/or persistent storage of any type, including, but not limited to, volatile and non-volatile memory, optical, magnetic, and/or solid state mass storage, and so forth. Volatile memory may include, but is not limited to, static and/or dynamic random-access memory. Non-volatile memory may include, but is not limited to, electrically erasable programmable read-only memory, phase change memory, resistive memory, and so forth.

The computing device 1300 may further include input/output (I/O) devices 1308 (such as a display, soft keyboard, touch sensitive screen, image capture device, and so forth) and communication interfaces 1310 (such as network interface cards, modems, infrared receivers, radio receivers (e.g., Near Field Communication (NFC), Bluetooth, WiFi, 4G/5G LTE), and so forth).

The communication interfaces 1310 may include communication chips (not shown) that may be configured to operate the device 1300 in accordance with a Global System for Mobile Communication (GSM), General Packet Radio Service (GPRS), Universal Mobile Telecommunications System (UMTS), High Speed Packet Access (HSPA), Evolved HSPA (E-HSPA), or Long-Term Evolution (LTE) network. The communication chips may also be configured to operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chips may be configured to operate in accordance with Code Division Multiple Access (CDMA), Time Division Multiple Access (TDMA), Digital Enhanced Cordless Telecommunications (DECT), Evolution-Data Optimized (EV-DO), derivatives thereof, as well as any other wireless protocols that are designated as 3G, 4G, 5G, and beyond. The communication interfaces 1310 may operate in accordance with other wireless protocols in other embodiments.

The above-described computing device 1300 elements may be coupled to each other via system bus 1312, which may represent one or more buses. In the case of multiple buses, they may be bridged by one or more bus bridges (not shown). Each of these elements may perform its conventional functions known in the art. In particular, system memory 1304 and mass storage devices 1306 may be employed to store a working copy and a permanent copy of the programming instructions implementing the operations associated with the wearable sensor apparatus 100, such as wireless stack and scheduler 308, signal processing module 310, and sensor interface 306 described in reference to the digital front end node 192 of FIG. 3. The various elements may be implemented by assembler instructions supported by processor(s) 1302 or high-level languages that may be compiled into such instructions.

The permanent copy of the programming instructions may be placed into permanent storage devices 1306 in the factory, or in the field, through, for example, a distribution medium (not shown), such as a compact disc (CD), or through communication interface 1310 (from a distribution server (not shown)). That is, one or more distribution media having an implementation of the agent program may be employed to distribute the agent and to program various computing devices.

The number, capability, and/or capacity of the elements 1308, 1310, 1312 may vary, depending on whether computing device 1300 is used as a stationary computing device, such as a set-top box or desktop computer, or a mobile computing device, such as a tablet computing device, laptop computer, game console, or smartphone. Their constitutions are otherwise known, and accordingly will not be further described.

At least one of processors 1302 may be packaged together with computational logic 1322 configured to practice aspects of embodiments described in reference to FIGS. 1-12. For one embodiment, at least one of processors 1302 may be packaged together with memory having computational logic 1322 to form a System in Package (SiP) or a System on Chip (SoC). For at least one embodiment, the SoC may be utilized in, e.g., but not limited to, a mobile computing device such as a computing tablet or smartphone, such as external device 184 of FIG. 1 or 520 of FIG. 5. In another embodiment, the SoC may be utilized to form the digital front end node 192 of FIGS. 1 and 3.

In embodiments, the computing device 1300 may associate with a wearable sensor apparatus 100 as described above. In some embodiments, the apparatus 100 may include sensor module 102, sensor front end module 142, and digital front end node 192 and may be communicatively coupled with the external device 184 implemented as computing device 1300 described herein.

In various implementations, the computing device 1300 may comprise a laptop, a netbook, a notebook, an ultrabook, a smartphone, a tablet, a personal digital assistant (PDA), an ultra mobile PC, a mobile phone, or a digital camera. In further implementations, the computing device 1300 may be any other electronic device that processes data.

Example 1 is a wearable apparatus for sensor measurements, comprising: a first flexible substrate attachable to a user's body, wherein the first flexible substrate includes a first plurality of sensors disposed on a first side of the first flexible substrate to be in direct contact with the user's body, and a first set of conductive connectors disposed on a second side of the first flexible substrate; and a second flexible substrate that includes a second set of conductive connectors disposed on a first side of the second flexible substrate compatibly to the first set of conductive connectors to mechanically and electrically couple the second flexible substrate with the first flexible substrate, and circuitry disposed on a second side of the second flexible substrate to receive and process readings provided by the first plurality of sensors via the first and second sets of conductive connectors.

Example 2 may include the subject matter of Example 1, wherein the first flexible substrate further includes: a second plurality of sensors disposed on the first side of the flexible substrate; and a third set of conductive connectors disposed on the first side of the flexible substrate to mechanically and electrically couple the second plurality of sensors with the first flexible substrate, wherein the circuitry is further to receive and process readings provided by the second plurality of sensors via the first, second, and third sets of conductive connectors.

Example 3 may include the subject matter of Example 2, wherein the first plurality of sensors comprises washable devices, and wherein the second plurality of sensors comprises non-washable devices.

Example 4 may include the subject matter of Example 2, wherein the first plurality of sensors includes at least selected ones of: optical photodiodes, electrocardiogram (ECG) electrodes, electromyogram (EMG) electrodes, galvanic skin response (GSR) electrodes, piezo crystals, pressure sensors, or stretch sensors.

Example 5 may include the subject matter of Example 2, wherein the second plurality of sensors includes at least selected ones of: temperature sensors, sweat chemical sensors, or motion sensors.

Example 6 may include the subject matter of Example 2, wherein the first, second, and third sets of conductive connectors comprise a flexible stretchable substrate, including at least a selected one of: elastic fabric, elastomer, or polymer.

Example 7 may include the subject matter of Example 1, wherein the second flexible substrate further includes one or more sensors disposed on the second side of the second flexible substrate.

Example 8 may include the subject matter of Example 1, wherein the circuitry includes a sensor front end module to excite the first plurality of sensors and to pre-process signals comprising readings provided by the first plurality of sensors, wherein to pre-process includes to transduce, amplify, and condition the signals.

Example 9 may include the subject matter of Example 8, wherein the circuitry further includes a digital front end node communicatively coupled with the sensor front end module to convert and further process the pre-processed signals provided by the sensor front end module.

Example 10 may include the subject matter of Example 9, wherein the digital front end node is to provide the processed signals to an external aggregating device for further processing.

Example 11 may include the subject matter of Example 1, wherein the first flexible substrate comprises a first strap of a washable flexible material.

Example 12 may include the subject matter of Example 11, wherein the second flexible substrate comprises a second strap of a flexible material that is conformable to the first strap.

Example 13 may include the subject matter of any of Examples 1 to 10, wherein the first flexible substrate further comprises a first wiring arrangement embedded in the first flexible substrate to provide electric coupling between the first and second sets of conductive connectors and between the first plurality of sensors and the first set of conductive connectors.

Example 14 may include the subject matter of Example 12, wherein the first wiring arrangement includes a selected one of: conductive traces disposed inside the first flexible substrate or screen-printed in the first flexible substrate, conductive threads woven or embroidered on the first flexible substrate, or conductive patterns deposited on a third substrate, wherein the third substrate is moulded with the first flexible substrate.

Example 15 may include the subject matter of Example 14, wherein the second flexible substrate further comprises a second wiring arrangement disposed inside the second flexible substrate to route electric signals between the second set of conductive connectors and the circuitry.

Example 16 is a wearable strap system, comprising one or more apparatuses that include: a first flexible substrate attachable to a user's body, wherein the first flexible substrate includes a first plurality of sensors disposed on a first side of the first flexible substrate to be in direct contact with the user's body, and a first set of conductive connectors disposed on a second side of the first flexible substrate; and a second flexible substrate that includes a second set of conductive connectors disposed on a first side of the second flexible substrate compatibly to the first set of conductive connectors to mechanically and electrically couple the second flexible substrate with the first flexible substrate, and circuitry disposed on a second side of the second flexible substrate to receive and process readings provided by the first plurality of sensors via the first and second sets of conductive connectors.

Example 17 may include the subject matter of Example 16, wherein the first flexible substrate further includes a second plurality of sensors disposed on the first side of the flexible substrate; and a third set of conductive connectors disposed on the first side of the flexible substrate to mechanically and electrically couple the second plurality of sensors with the first flexible substrate, wherein the circuitry is further to receive and process readings provided by the second plurality of sensors via the first, second, and third sets of conductive connectors, wherein the first plurality of sensors comprises washable devices, and wherein the second plurality of sensors comprises non-washable devices.

Example 18 may include the subject matter of any of Examples 16 to 17, wherein the circuitry includes a sensor front end module to excite the first plurality of sensors and to pre-process signals comprising readings provided by the first plurality of sensors, and a digital front end node communicatively coupled with the sensor front end module to convert and further process the pre-processed signals provided by the sensor front end module and to provide the processed signals to an external aggregating device for further processing.

Example 19 may include the subject matter of Example 18, wherein the first and second flexible substrates comprise first and second wearable straps, wherein the one or more apparatuses include one or more of: a wearable knee strap apparatus, a wearable chest strap apparatus, a wearable neck strap apparatus, a wearable wrist strap apparatus, or a wearable foot strap apparatus, wherein the external aggregating device comprises a mobile computing device.

Example 20 is a method for providing a wearable apparatus for sensor measurements, comprising: disposing a first plurality of sensors on a first side of a first flexible substrate to be in direct contact with a user's body; disposing a first set of conductive connectors on a second side of the first flexible substrate; disposing a second set of conductive connectors on a first side of a second flexible substrate compatibly to the first set of conductive connectors to mechanically and electrically couple the second flexible substrate with the first flexible substrate; and disposing circuitry on a second side of the second flexible substrate to receive and process readings provided by the first plurality of sensors via the first and second sets of conductive connectors.

Example 21 may include the subject matter of Example 20, further comprising: disposing a third set of conductive connectors on the first side of the first flexible substrate; and attaching a second plurality of sensors to the first side via the third set of conductive connectors, wherein the circuitry is to receive and process readings provided by the second plurality of sensors via the first, second, and third sets of conductive connectors.

Example 22 may include the subject matter of Example 21, wherein disposing circuitry includes: disposing a sensor front end module to excite the first plurality of sensors and pre-process signals comprising readings provided by the first and second plurality of sensors.

Example 23 may include the subject matter of Example 22, further comprising: communicatively coupling a digital front end node with the sensor front end module, to convert and further process the pre-processed signals provided by the sensor front end module.

Example 24 may include the subject matter of Example 20, further comprising: disposing a first wiring arrangement inside the first flexible substrate to provide electric coupling between the first and second sets of conductive connectors and between the first plurality of sensors and the first set of conductive connectors.

Example 25 may include the subject matter of any of Examples 20 to 24, further comprising: disposing a second wiring arrangement inside the second flexible substrate to route electric signals between the second set of conductive connectors and the circuitry

Various operations are described as multiple discrete operations in turn, in a manner that is most helpful in understanding the claimed subject matter. However, the order of description should not be construed as to imply that these operations are necessarily order dependent. Embodiments of the present disclosure may be implemented into a system using any suitable hardware and/or software to configure as desired.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims and the equivalents thereof.

What is claimed is:

1. An apparatus, comprising:
a first flexible substrate attachable to a user's body, wherein the first flexible substrate includes a first plurality of sensors disposed on a first side of the first flexible substrate to be in direct contact with the user's body, and a first set of conductive connectors disposed on a second side of the first flexible substrate; and
a second flexible substrate that includes a second set of conductive connectors disposed on a first side of the second flexible substrate compatibly to the first set of conductive connectors to mechanically and electrically couple the second flexible substrate with the first flexible substrate, and circuitry disposed on a second side of the second flexible substrate to receive and process readings provided by the first plurality of sensors via the first and second sets of conductive connectors.

2. The apparatus of claim 1, wherein the first flexible substrate further includes:
a second plurality of sensors disposed on the first side of the flexible substrate; and
a third set of conductive connectors disposed on the first side of the flexible substrate to mechanically and electrically couple the second plurality of sensors with the first flexible substrate, wherein the circuitry is further to receive and process readings provided by the second plurality of sensors via the first, second, and third sets of conductive connectors.

3. The apparatus of claim 2, wherein the first plurality of sensors comprises washable devices, and wherein the second plurality of sensors comprises non-washable devices.

4. The apparatus of claim 2, wherein the first plurality of sensors includes at least selected ones of: optical photodiodes, electrocardiogram (ECG) electrodes, electromyogram (EMG) electrodes, galvanic skin response (GSR) electrodes, piezo crystals, pressure sensors, or stretch sensors.

5. The apparatus of claim 2, wherein the second plurality of sensors includes at least selected ones of: temperature sensors, sweat chemical sensors, or motion sensors.

6. The apparatus of claim 2, wherein the first, second, and third sets of conductive connectors comprise a flexible stretchable substrate, including at least a selected one of: elastic fabric, elastomer, or polymer.

7. The apparatus of claim 1, wherein the second flexible substrate further includes one or more sensors disposed on the second side of the second flexible substrate.

8. The apparatus of claim 1, wherein the circuitry includes a sensor front end module to excite the first plurality of sensors and to pre-process signals comprising readings provided by the first plurality of sensors, wherein to pre-process includes to transduce, amplify, and condition the signals.

9. The apparatus of claim 8, wherein the circuitry further includes a digital front end node communicatively coupled with the sensor front end module to convert and further process the pre-processed signals provided by the sensor front end module.

10. The apparatus of claim 9, wherein the digital front end node is to provide the processed signals to an external aggregating device for further processing.

11. The apparatus of claim 1, wherein the first flexible substrate comprises a first strap of a washable flexible material.

12. The apparatus of claim 11, wherein the second flexible substrate comprises a second strap of a flexible material that is conformable to the first strap.

13. The apparatus of claim 1, wherein the first flexible substrate further comprises a first wiring arrangement embedded in the first flexible substrate to provide electric coupling between the first and second sets of conductive connectors and between the first plurality of sensors and the first set of conductive connectors.

14. The apparatus of claim 12, wherein the first wiring arrangement includes a selected one of: conductive traces disposed inside the first flexible substrate or screen-printed in the first flexible substrate, conductive threads woven or embroidered on the first flexible substrate, or conductive patterns deposited on a third substrate, wherein the third substrate is moulded with the first flexible substrate.

15. The apparatus of claim 14, wherein the second flexible substrate further comprises a second wiring arrangement disposed inside the second flexible substrate to route electric signals between the second set of conductive connectors and the circuitry.

16. A wearable strap system, comprising one or more apparatuses that include:
a first flexible substrate attachable to a user's body, wherein the first flexible substrate includes a first plurality of sensors disposed on a first side of the first flexible substrate to be in direct contact with the user's body, and a first set of conductive connectors disposed on a second side of the first flexible substrate; and
a second flexible substrate that includes a second set of conductive connectors disposed on a first side of the second flexible substrate compatibly to the first set of conductive connectors to mechanically and electrically couple the second flexible substrate with the first flexible substrate, and circuitry disposed on a second side of the second flexible substrate to receive and process readings provided by the first plurality of sensors via the first and second sets of conductive connectors.

17. The wearable strap system of claim 16, wherein the first flexible substrate further includes a second plurality of sensors disposed on the first side of the flexible substrate; and a third set of conductive connectors disposed on the first side of the flexible substrate to mechanically and electrically couple the second plurality of sensors with the first flexible substrate, wherein the circuitry is further to receive and process readings provided by the second plurality of sensors via the first, second, and third sets of conductive connectors, wherein the first plurality of sensors comprises washable devices, and wherein the second plurality of sensors comprises non-washable devices.

18. The wearable strap system of claim 16, wherein the circuitry includes a sensor front end module to excite the first plurality of sensors and to pre-process signals comprising readings provided by the first plurality of sensors, and a digital front end node communicatively coupled with the sensor front end module to convert and further process the pre-processed signals provided by the sensor front end module and to provide the processed signals to an external aggregating device for further processing.

19. The wearable strap system of claim 18, wherein the first and second flexible substrates comprise first and second wearable straps, wherein the one or more apparatuses include one or more of: a wearable knee strap apparatus, a wearable chest strap apparatus, a wearable neck strap apparatus, a wearable wrist strap apparatus, or a wearable foot strap apparatus, wherein the external aggregating device comprises a mobile computing device.

20. A method, comprising:
disposing a first plurality of sensors on a first side of a first flexible substrate to be in direct contact with a user's body;
disposing a first set of conductive connectors on a second side of the first flexible substrate;
disposing a second set of conductive connectors on a first side of a second flexible substrate compatibly to the first set of conductive connectors to mechanically and electrically couple the second flexible substrate with the first flexible substrate; and
disposing circuitry on a second side of the second flexible substrate to receive and process readings provided by the first plurality of sensors via the first and second sets of conductive connectors.

21. The method of claim 20, further comprising:
disposing a third set of conductive connectors on the first side of the first flexible substrate; and
attaching a second plurality of sensors to the first side via the third set of conductive connectors, wherein the circuitry is to receive and process readings provided by the second plurality of sensors via the first, second, and third sets of conductive connectors.

22. The method of claim 21, wherein disposing circuitry includes:
disposing a sensor front end module to excite the first plurality of sensors and pre-process signals comprising readings provided by the first and second plurality of sensors.

23. The method of claim 22, further comprising:
communicatively coupling a digital front end node with the sensor front end module, to convert and further process the pre-processed signals provided by the sensor front end module.

24. The method of claim 20, further comprising:
disposing a first wiring arrangement inside the first flexible substrate to provide electric coupling between the first and second sets of conductive connectors and between the first plurality of sensors and the first set of conductive connectors.

25. The method of claim 20, further comprising:
disposing a second wiring arrangement inside the second flexible substrate to route electric signals between the second set of conductive connectors and the circuitry.
